(19) United States
(12) Patent Application Publication
(10) Pub. No.: US 2023/0154263 A1
(43) Pub. Date: May 18, 2023

(54) SYSTEM AND METHOD FOR BUILDING ENTRY MANAGEMENT SYSTEM

(71) Applicant: Repticity Inc., Brooklyn, NY (US)

(72) Inventor: Shrage Toiv, Brooklyn, NY (US)

(21) Appl. No.: 17/995,588

(22) PCT Filed: Apr. 6, 2021
(86) PCT No.: PCT/US2021/026011

§ 371 Date: Oct. 6, 2022

Related U.S. Application Data
(60) Provisional application No. 63/006,713, filed on Apr. 7, 2020.

(51) Int. Cl. G07C 9/28; G07C 9/27; G16H 50/30; G16H 50/80

(52) U.S. Cl. CPC G07C 9/28; G07C 9/27; G16H 50/30; G16H 50/80

(57) ABSTRACT

A method for and system for managing building entry is provided. The method includes receiving from a visitor identification information; determining geographical area of the building and obtaining local health information related to the geographical area; obtaining medical data including at least one vital sign of the visitor; calculating risk level of the visitor, based on the medical data and the local health information; and determining building access for the visitor based on the calculated risk level for the visitor.

### TECHNICAL FIELD

The presently disclosed subject matter relates to a system and method for building entry management.

### BACKGROUND OF THE INVENTION

There are known building entry management systems which aim to facilitate visitor and guest management. These building entry management systems collect data of visitors, and based on the collected data, provide or deny access to a party seeking entry to the building.

### SUMMARY OF INVENTION

There is provided in accordance with an aspect of the presently disclosed subject matter a method for managing building entry. The method includes receiving from a visitor identification information; determining geographical area of the building and obtaining local health information related to said geographical area; obtaining medical data including at least one vital sign of the visitor; calculating risk level of the visitor, based on the medical data and the local health information; and determining building access for the visitor based on the calculated risk level for the visitor.

The at least one vital sign can be selected from a group including body temperature, blood pressure, heart rate, respiratory rate, and oxygen level.

The step of obtaining medical data can further include prompting the visitor to input data related to health condition.

The method can further include obtaining locations data related to physical locations of the visitor during a predetermined period prior to the visit, and wherein the step of calculating risk level can be carried out in accordance with the locations data.

Obtaining the locations data can include receiving location information from a handheld device of the visitor.

Obtaining the medical data can include receiving an image of the visitor and extracting from the image the at least one vital sign.

The method can further include determining visit characteristics of a visit by the visitor, the visit characteristics including a visit purpose. The visit characteristics can include data related to sections of the building to be visited during the visit.

The step of providing the visitor with a building access information can include sending the access information to a handheld device of a user, wherein the access information is encoded.

The access information can include access grant or denial and access restriction.

The method can further include forming a visit log including the visitor identification information, visit characteristics, and access information.

The step of calculating risk level can include obtaining information from visit logs of previous visits and calculating risk level of the visit in accordance with risk levels of the previous visits.

The step of calculating risk level can include obtaining information from visit logs of previous visits and calculating risk level of the visit in accordance with risk levels of the previous visits.

The method can further include tracking visiting locations of the visitor in the building during the visit and recording data of the visiting locations in the visit log.

There is provided in accordance with another aspect of the presently disclosed subject matter a system for managing building entry. The system includes a plurality of input devices configured for inputting identification information of a visitor and medical data related to at least one vital sign of the visitor; an infection data module configured for receiving health information related to geographical area of a building to be visited; and an access controller server configured to receive the identification information and the medical data, the access controller server is configured for calculating risk level of the visit in accordance with the medical data and the health information, the access controller server is further configured for providing the visitor with a building access information determined in accordance with the risk level.

The risk level may change for a given time period based, for example, on data received/obtained from local health authorities, such as the Centers for Disease Control and Prevention (CDC), or other databases collecting data related to community infection rate. Based on this data, the risk threshold value for safe entry may be greater or less than it was at a previous time.

The system can further include a medical diagnostic tool for obtaining the at least one vital sign, and wherein the at least one vital sign is selected from a group including body temperature, blood pressure, heart rate, respiratory rate, and oxygen level.

The system can be further configured to determine visit characteristics of a visit by the visitor, the visit characteristics including a visit purpose, and wherein the calculating risk level can be carried out in accordance with the characteristics.

The plurality of input devices can be configured for obtaining locations data related to physical locations of the visitor during a predetermined period prior to the visit, and wherein the access controller server is configured for calculating risk level in accordance with the locations data.

The input devices can be configured for obtaining an image of the visitor, and wherein the system can further include an image analyzer configured to extract from the image vitals of the visitor.

The building access information can include encoded information related to access restriction.

The system can further include wearable electronic devices configured for tracking contacts of the visitor inside the building.

As used herein the specification and claims visitor may mean any person entering a building or facility for any purpose including employees entering the building or facility as part of their routine employment responsibilities, vendors entering the building or facility for delivering supply or providing services, and person entering the building for the purpose of visiting or assisting another person residing in the building or facility.

As used herein the specification and claims “visit” may mean any entry into the building or facility for any purpose including entering the building or facility for carrying out employee responsibility, entering the building or facility for delivering supply or providing services, and entering the building or facility for visiting or assisting another person residing in the building or facility.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1 is block diagram of a system for building entry management in accordance with an example of the presently disclosed subject matter; and,

FIG. 2 is a flow chart illustration of a method for building entry management in accordance with an example of the presently disclosed subject matter.

### DESCRIPTION OF DRAWING FIGURES

The presently disclosed subject matter is directed to a building entry management system for analyzing medical conditions of persons seeking entry into the building, premises, or the like, and, based on the risk caused by these medical conditions, granting or denying access to the building. The system allows monitoring and precluding the spread of viruses and other contagious diseases within the building.

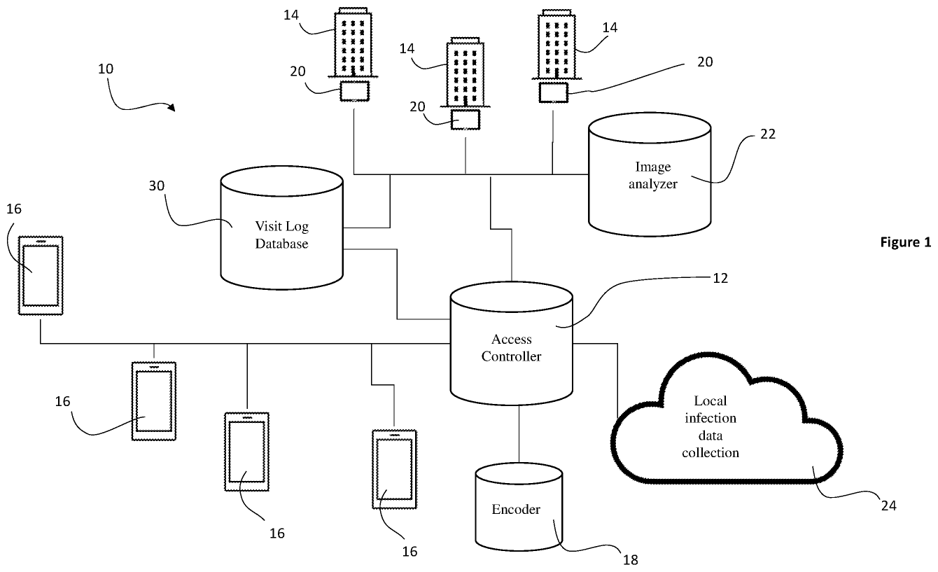

As shown in FIG. 1, the system 10 includes an access controller server 12, which can be accessed by a plurality of remote facilities, represented by their computer systems 14, in communications with the access control server 12. The computer systems include, for example, those of old age homes, assisted living facilities, hospitals, or other facilities, which are both public and private. The system 10 further includes a plurality of handheld devices 16 which, communicate with the access control server 12, and, for example, include smartphones carrying and running a designated software application, or having access to a designated web interface. The access controller server 12 is configured to receive data regarding a future visit, to analyze and determine risks associated with the expected visit and to provide an access decision. The access decision maybe granting or denying access, and can further include information regarding restrictions or conditions for the expected visit.

According to an example, the access controller server 12 receives information regarding the expected visit from one of the handheld devices 16, for example, information inputted by a visitor wishing to enter one of the remote facilities 14. The user, is, for example, prompted to enter information related to the remote facility 14 he intends to visit, and to answer a series of questions related to his/her health condition and other information that may affect the safety of the visit to the facility. The access controller server 12 is then configured to analyze the received data, and calculate the risk involved with the future visit and to send the access decision to the handheld device 16 held by the visitor as well to the remote facility 14 to be visited. Should the calculated risk exceed a predetermined threshold value (e.g., a minimum threshold value), access is denied, or referred to a system administrator, or other administrative entity, to decide on access. Should the calculated risk be less than a predetermined threshold value (e.g., a minimum threshold value), access to the person seeking entry is granted. While a single person seeking entry is described, the system can also be operated for a group of people, e.g., two or more, seeking entry to the building, facility, or the like.

According to an example, the system 10 further incudes an encoder 18 which is configured to encode the access decision and to provide the visitor with a code, such as a Quick Response (QR) code, which can be verified by the facility to be visited. According to this example, the remote facilities 14 are provided with an input device 20, which can be configured to scan the QR code, validate the encoded information and indicate whether or not the visitor is granted access to the facility. The input device 20 can be a computer kiosk disposed at the entrance of the facility and in communication with the access controller server 12. The input device 20, for example, is further be configured for input of data, such as health data inputted by the visitor. According to an example, the input device 20 can be coupled to a medical device for obtaining vitals of the visitors, such as a thermometer or other medical devices for obtaining the body temperature of the user, blood pressure monitor, pulse/heart rate monitor, or the like. This way, while the visitor provides information regarding the expected visit via the handheld device 16, more up to date information can be obtained via the input device 20 at the entrance to the facility 14 immediately prior to the entrance.

According to a further example, the input devices 20 and/or the handheld devices 16 can be configured to obtain an image of the visitor, such as an image of his/her face or hand. The image is then sent to an image analyzer 22 which is configured to extract from the image vitals and/or vital signs of the visitor. The image analyzer 22, for example, is equipped with an image processor configured for real-time vital signs measurements in accordance with an image provided by a visitor, such as disclosed by https://www.binah.ai/. Accordingly, the system 10 allows obtaining an image of a visitor entering the facility and extracting real time vital of the visitor, based on which access to the facility can be granted or denied.

Otherwise, the vitals can be manually inputted via the handheld device 16, or via thermometer, blood pressure device, pulse/heart rate monitor, or the like, coupled to the handheld device 16.

According to an example, the system 10 can be configured to telemedicine validation. For example, in case the system detects a parameter which may indicate that the visitor may be at risk, the handheld device 16 can initiate communication with remote medical personnel, who can validate the inputted information, or provide further information for the risk calculation.

Furthermore, the input devices 20 and/or the handheld devices 16 can be configured to validate the identity of the visitor by facial recognition, which can be carried out at the entrance to the facility and/or when information is inputted on the handheld device. This way, the system 10 can preclude errors or possible fraud including providing false medical information or in a case on an employee, checking in for a co-employee.

. Similarly, the system can require two factor authentication (e.g., an identification of a visitor device and an identifier, such as a personal identification (PIN) code, or other security feature, when entering medical information via the handheld devices 16.

Moreover, according to an example, the system 10 can be configured for collaborating with time and attendance systems, for tracking employees. This way, the management system 10, can be configured to receive notification when an employee clocks-in on the attendance systems, and to verify that the employee has input his/her health information and was granted access. The system can be further configured to alert an employee when a clock-in is detected and no access was granted by the system 10. Such alert can be sent as a text or email message to the employee and his manager.

According to another example, the system 10 can further include an infection data module 24 for receiving health data in a predetermined geographical area in which the facility to be entered is located. For example, the infection data module 24 can be configured to receive data from local health authorities, such as the Centers for Disease Control and Prevention (CDC), or other databases collecting data related to community infection rate. The system 10 and in particular the access controller server 12 can consider data received from the infection data module 24 for calculating the risk level involved in the future visit, or for a predetermined time period, such that the risk level, e.g., risk score threshold, is different at different time periods, based, for example, on community infection rate data. For example, in case of visit of a facility located in a city in which a high infection rate is reported, the risk level of the visit can be adapted to a higher risk regardless of the health condition of the visitor. For example, should the area within a predetermined radius around the facility, be “red”, meaning a high virus (e.g., COVID) area, access can be denied to all, outright, and limited to just residents of a building or essential personnel of a facility, for example, due to a low (minimum) threshold value, which the risk score exceeds. Should, within a day or two later (e.g., another time period or predetermined time period), the area within the predetermined radius around the facility be “yellow”, meaning a low virus rate, the risk threshold score may be raised, so that non-residents who meet the health criteria and have risk scores under the risk score threshold.

The system 10 can be configured to receive data from a plurality of handheld devices to analyze trends in certain areas, so as to mitigate the risk of allowing access to a visitor infected by a virus, however without having symptoms, known as an asymptomatic carrier.

According to an example, the system 10 can be configured to collect data of visitors over a certain period of time. The access decision can be made in accordance with the accumulative data of the visitor's physical condition. For example, if a potential visitor is detected as having fever, the visitor may be denied access to the building for the next three full days, even if on the following day the visitor has no fever, as recommended by the Centers for Disease Control and Prevention (CDC), or other authority. This way, the building entry management system can track medical conditions of hundreds of visitors, and provide a more efficient and safe access decision in accordance with the recorded medical conditions of each visitor over a predetermined period of time.

The server 12 may be coupled to other sources of data such as sources that aggregate data from people using the Kinsa Bluetooth™ thermometer and data from multiple sources to develop a highly accurate long-lead forecast. Such sources can be, for example, https://content.kinsahealth.com/covid-detection-technical-approach and https://healthweather.us/ the content of which is incorporated herein by reference. Likewise, the server 12 can be coupled to other data sources providing real-time data related to risk of various spread, such as https://covidactnow.org/?s=1672604 providing data related to risk of Covid-19 depending on geographical area.

Data from these sources can be utilized for screening visitors from areas having a high likelihood of being infected with a virus, such as COVID-19 (COVID).

More specifically, in case of a nursing home, the server 12 can be coupled to a data source, for example, a computer system and/or server of a local health authority, providing infection rates in other nursing homes in the area. For example, haps://data.cms.gov/stories/s/COVID-19-Nursing-Home-Data/bkwz-xpvg/ which allow receiving and analyzing data related to infection rates in nursing homes. This specific data can be utilized for screening employees or vendors accessing multiple nursing homes on a regular basis. This way, in case one nursing home is reported as having a high infection rate, any employee working at this nursing home can be denied access to other nursing homes or be provided with requirements and restrictions for accessing other nursing homes.

Furthermore, according to another example, the infection data module 24 can be configured to automatically receive guidelines and health ordinances issued by local authorities with respect to facilities in the geographical area in which the facility to be visited in located. For example, if local authorities restrict entrance to certain facilities for a certain period of time, such as entering old age homes during a local disease outbreak, the system 10 can be configured to deny access to any facility in the area.

Moreover, the system 10 can include artificial intelligence capabilities allowing the system to detect and collect real-time regulatory information pertaining to each facility. For example, the system 10 can be configured to receive data from local health authorities of multiple counties and states and adapt the visit rules and conditions in accordance with these rules. The system can include multiple links to online informational sources published by health authorities, and detect visit regulations pertaining to each facility. For example, should the system detect new guidelines pertaining to nursing homes in a certain county, the infection data module 24 can be configured to update the visitation rules and restrictions for nursing home located in this county.

Similarly, in case regulation updates are sent to nursing homes, or similar facilities, via email or other electronic communication formats, including social networks, the system 10 can include artificial intelligence capabilities to detect such as electronic communications, and extract the relevant information for visitation rules.

In addition, in case guidelines require verification of certain medical records of the visitor prior to entering a facility, the system 10 can be further configured to access document management systems, or medical record systems. This way, if for example, guidelines require visitors to certain facilities to be vaccinated against a certain virus, the system 10 can access the medical records of the visitor and verify data related to the required vaccine. Similarly, if guidelines require visitors to be tested for active virus infection prior to their visit, the system 10 can access the medical records of the visitor to verify the test results.

Alternatively, the system 10 can be configured to verify medical documents, such as a document including various test result. This can be carried out by allowing the visitor to upload or scan medical records and verifying the uploaded document by using OCR type technology. For example, COVID vaccination and recovery certificates may be verified in this manner.

This data can be used for ensuring compliance with local health guidelines or as part of the risk assessments. For example, the days past since the last virus test can be included in the calculation of the risk associated with the expected visit.

The access controller server 12 can be further configured to receive from the handheld devices 16 information regarding locations of the visitor prior to the visit. Such locations can be obtained by a location tracker embedded in the handheld devices 16. The location tracker can be configured to share location information of locations visited by the visitor within a predetermined period of time prior to his/her visit to the facility. This way, the access controller server 12 can further consider risk level in accordance with number and nature of locations in which the visitor visited prior to his/her visit. This location data can be considered in conjunction with the data received from the infection data module 24. For example, if the visitor visited an area in which infection outbreak was reported, the risk level associated with the visit can be adapted accordingly.

Moreover, handheld devices 16, are for example, wearable devices, which are configured to track virus symptoms, such as body temperature, heart rate, oxygen level, and the like, using such a device, which is, example described in: https://www.samsung.com/us/watches/galaxy-watch3/#health, the disclosure of which is incorporated by reference herein.

This way, the user's medical condition is constantly being monitored, facilitating thereby detecting long-term trends in the visitor's medical condition. The access decision can be made in accordance with the medical conditions and trends provided by the wearable device.

According to a further example, the input devices 20 can be devices disposed at the entrance to the building and can be configured to collect data from the visitor's handheld devices 16. The visitor can be prompted to input information on his/her personal device and the information is transmitted to the port as soon as the visitor enters the building. The input devices 20 and the personal device can be configured for contactless communication, so as to reduce the physical contact between visitors and preclude virus spread. Contactless communication for handheld devices is known, for example: haps://developer.apple.com/design/human-interface-guidelines/ios/user-interaction/near-field-communication/.

Finally, the system 10 can include a visit log database 30, which is in communication with the access controller server 12 and the facility's server or computer system 14. The visit log database 30, is, for example, configured to collect information regarding previous visits to each facility, including the time and date of the visit, the visitor's information, the risk level associated with the visit, as calculated by the access controller server 12. The visit log can be used by the system 10 to assess possible infections when a past visitor was found to be infected. In addition, the visit log can be used for self-learning of the access controller server 12, for example, by assessing risk levels of past visits in a certain facility and infection rates in the facility following the visit. Finally, the information from the access controller server 12 can be used for the risk level calculation of visits. For example, in case the visit logs indicate that a relatively high number of high-risk visitors are scheduled to visit a facility on a certain day, the access controller server 12 can be configured to grant further access on the same day on to low-risk visitors.

According to an example, the log database 30 can be configured to collect medical data of each visitor, such that over time, a baseline for each medical parameter can be established. This way, the system 10 can detect variations in the baseline and improve the detection of possible infection of the visitor and thus improve the risk assessment.

The above-described system can be used for managing entrance to various facilities, such as nursing homes, hospitals, or other types of medical institutions. According to another example, the system can be configured to facilitate entrance to other facilities such as schools, office buildings, shopping malls, airports, train stations, and the like. The system can be configured for real-time analyzing and determining safety of entrance of each expected visitor. The system can thus be used for considering multiple parameters pertaining to the health and infection risk of each visitor and provide an access code allowing entrance to the facility.

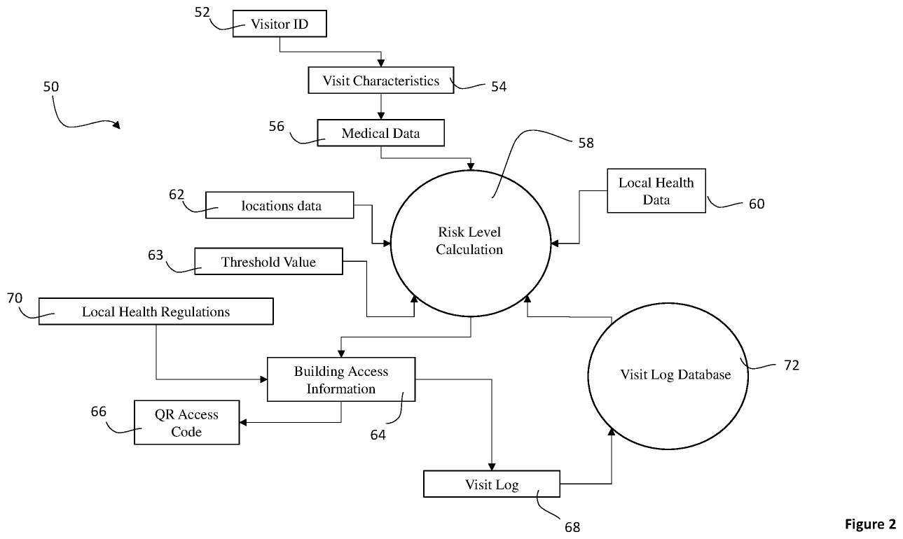

Reference is now made to FIG. 2, illustrating a method 50 for managing building entry, for example, by the system 10 of FIG. 1. The method 50 includes, for example, receiving identification information of the visitor (block 52). The identification information can include: name, address and contact information of the visitor. In addition, the identification information can include visitor's age and preexisting medical conditions which may affect the access to the facility. The information can be inputted via a computer kiosk at the entrance to the facility to be visited, or by via a handheld device held by the visitor.

According to an example, visitors can enroll in the building entry system and be provided with a user-name of other identification information. This way, receiving identification information of a user, can be receiving a visit request from a preregistered handheld device associated with a certain user. When such visit request is received the building entry system can retrieve the pre-stored user's information.

The method 50 further includes determining visit characteristics of the intended visit (block 54), such as the visit purpose, for example, vendor entering the building to facility to deliver supply, an employee who works at the facility, or a visitor intending to visit a resident of the facility. The visit characteristics can further include determining areas of the building which are intended to be visited during the visit. The building entry system can be configured to determine the purpose of visit and areas to be visited in accordance with the pre-registered information. For example, if a vendor enters the building to deliver supply to a kitchen in the facility, determining visit characteristics can be carried out by identifying the visitor and identifying the kitchen as a pre-stored area to be visited by the vendor.

The method 50 further includes receiving medical data related to at least one vital sign of the visitor (block 56). The vital sign can include one or more body vitals which can indicate whether or not the visitor is infected or is at a high risk to be infected. The vital signs can be body temperature, blood pressure, heart rate, respiratory rate, and/or oxygen level. According to an example the vital signs can be obtained by receiving an image of the visitor and extracting from the image the desired vital sign. Similarly, the vital signs can be obtained by a therapeutic diagnostic tool electronically coupled to a computer kiosk disposed at the entrance to the facility to be visited. For example, the therapeutic diagnostic tool can be a Bluetooth thermometer, such as KINSA thermometer, or WAND Blue device described here: https://www.thermoworks.com/wand-blue, which can be coupled to an app installed on the computer kiosk.

This way, upon entering the building, the visitor is prompted to measure his/her body temperature, which is then transmitted to the computer kiosk.

According to an example, obtaining the medical data can also include prompting the visitor to input data related to his/her health condition. For example, the user can be prompted to answer health question on his handheld device, or on a computer kiosk disposed at the entrance to the facility to be visited. The questions can be predetermined fixed questions directed to reveal any medical condition. According to an example, the data inputted by the visitor may be related to the visitor's medical condition, such as body temperature, heart rate, and other symptoms, which may indicate that the visitor is ill.

Alternatively, the health questions can include a dynamic set of questions, which are displayed to the visitor's in accordance with the risk level associated with the visit. For example, if the visitor is a vendor only entering a warehouse section of the facility, thus, presenting a relatively low risk, the health questions can be configured to reveal basic health information. If, on the other hand, the visitor intends to visit senior residence in the facility, and/or if the visitor present data which indicates a higher infection risk, the set of questions may include elaborated questions aimed to negate health risk.

Further, the method 50 includes calculating risk level of the intended visit (block 58) in accordance with the visit characteristics, i.e., visitor type and areas to be visited, and in accordance with the medical data received from the visitor. Calculating the risk level can be carried out by considering parameters related to the visit, such as possible people within the facility which may be in contact with the visitor during the visit, the medical condition of these people, and the health information of the visitor. Each parameter can be assigned a predetermined risk value, such that the risk calculation provides the total risk associated with the visit. This way, when the visit includes, for example, close contact with a senior residence of an old age home, the preset risk value of the visit characteristics is assigned a higher risk value, as opposed to a risk value of a visitor which only visits service areas at the old age home.

Similarly, when some of the medical data of the visitor indicates that he/she may be infected the risk value associated with the medical data may be higher than when the medical data does provide any indication for infection. Thus, calculating risk level is carried out by considering various parameters and the risk value associated with each parameter. This way, two visitors with similar medical data may be assigned different risk levels depending on the purpose of the visit and/or possible contacts throughout the intended visit.

According to an example, calculating the risk level can consider other parameters which may have an effect on the infection risk associated with future visit. For example, the method 50 may include determining geographical area of the building and obtaining health information related to the local health data (block 60). I.e., receiving data from local health authorities, such as the Centers for Disease Control and Prevention (CDC), or other databases collecting data related to community infection rate. The method thus includes calculating the risk level involved in the future visit of visitors from the infected area. For example, in case of visit of a facility located in a city in which a high infection rate is reported, the risk level of the visit can be adapted in accordance with the reported infection rate.

According to a further example, the method 50 may include obtaining locations data related to physical locations of the visitor during a predetermined period prior to visit (block 62). For example, the locations data can be obtained from the visitor's handheld device and can be considered in conjunction with health information related to the local health data (block 60). I.e., when health information indicates high infection rate in a certain area and the locations data of the visitor indicate visiting this area, the locations data may be given a certain risk value, which affects the overall calculated risk level.

According to an example, each parameter considered during the risk level is provided with a weighing value which provides the overall weight of the parameter with respect to other parameters. In other words, when considering the risk of possible contacts during the visit, the risk associated with the parameter can have a more significant weight with respect to risk associated with the other parameters, such as local health data. The weighing value of each parameter affects how much each parameter influences the overall calculated risk. The weighing values can be determined by facility manager or local regulations and may be adapted in accordance with the local requirements or the nature of the facility. The weighted values are calculated, for example, by a weighting algorithm, which determines a risk score, for the particular building.

According to an example, the weighted values can be determined in accordance with other parameters associated with the visitor. For example, if a visitor is an employee who works in another high risk facility, the weighing value assigned to vitals of this employee can be higher than weighing value assigned to vitals other visitors. This way, the overall risk calculation of this employee is more sensitive to changes in body vitals and the system provides a higher monitoring of access of this employee.

The analysis of these parameters and/or values, which may be on the order of hundreds or thousands, elements, to calculate a visitor's risk score, is performed, for example, contemporaneously, and may be performed simultaneously, and accordingly is a “big data” analysis, which is performed by special purpose computers. The “big data” analysis, for example, includes analyzing and systematically extracting information, or otherwise dealing with data sets that are too large or complex to be dealt with by traditional data-processing application software and/or software tools, for example, by specialized computer hardware and/or software, to capture, curate, manage, and process data within a tolerable elapsed time, for example, on the order of seconds, and, for example, in real time.

With the risk score calculated, it is compared to a predetermined threshold value (block 63) in order to grant or deny the person (e.g., visitor) entrance to the building, facility or the like. As the threshold value is predetermined, it may change over time, for example, should the area of the building be a code “red” or high virus contagion rate, the risk score threshold may be high, as compared to a code “yellow”, for low virus contagion rate, the risk score threshold may be low, for example, this threshold set by the system administrator or other person of authority, at any given time.

Once risk calculation is completed, the visitor is provided with the building access information (block 64), which is determined in accordance with the risk level. The building access information can include access grant or denial and can further include access restriction, such as restriction the areas to be visited or setting conditions to be fulfilled prior to the entrance, such as conducting a virus infection test. The building access information can be sent to the computer kiosk or the handheld device of the visitor. According to an example the building access information can be encoded (block 66), such that the visitor is provided with a QR code which can be validate by the computer kiosk at the facility to be entered.

Similarly, the building access information (block 64) can include generating a digital badge including the access conditions and restrictions. For example, the digital badge can be sent to the visitor's portable phone, and can be displayed in various colors, each color indicating access level, such as described here: https://emocha.com/resources-for-covid-19-return-to-work-service/. This way, the visitor can be granted access limited to certain areas of the facility depending on the risk level associated with his/her medical condition.

Similarly, the building access information (block 64), can include information regarding the type of protection required by the visitor. For example, a visitor who may have a higher risk, may be required to wear certain face/body covering with certain specifications. Thus, the risk assessment can include means for mitigating the risks by taking protective measures in accordance with the health parameters of the visitor and the risk level at the areas to be visited.

The system can be configured to alert the visitor via a text or email messages regarding any required entry conditions, such as “don't forget to wash your hands”, “mask covering is mandatory”, and the like.

Furthermore, the badge or the QR code can also be configured to provide the visitor with access to certain areas within the facility. For example, the facility can include barcode reader integrated with the door system of various areas, such as https://www.telpo.com.cn/mobile-pos/tps508-qr-code-pos.html. The visitor is then required to scan his access code to gain access to these areas and can be either granted or denied access in accordance with the determined building access information.

According to an example, the building access information (block 64) can be adapted in accordance with local health regulations (block 70). For example, building access information can include the access decision in accordance with the calculated risk but can further includes restrictions and conditions dictated by local health regulations. For example, if local health regulations require that certain visitor conduct a virus test prior to visiting a certain facility, regardless of the calculated risk, the access information is adapted to include the requirements under the local health regulations.

In addition, receiving local health regulations, can include receiving notifications from local authorities regarding visitation rules. For example, in case local authorities restrict all visitations to nursing homes for a certain period of time, this update can be automatically updated to the building entry management system such that the system can stop all unauthorized visitors from entering the nursing home.

Finally, the method includes recording data related to the visit in a visit log (block 68), which may include information regarding the visit, such as time and date, purpose of the visit, areas visited, the visitor's information, the risk level associated with the visit, as calculated prior to the visit.

According to an example, the step of calculating risk level (block 58) further includes considering visit logs of previous visits (block 72), which can be stored in a visit log database. For example, in case the visit logs indicate that a relatively high number of high-risk visitors are scheduled to visit a facility on a certain day, the grant of further access on the same day may be carried out considering the number of high-risk visitors.

The building entry management system and method can be configured to be integrated with other healthcare technology companies providing telemedicine services. For example, ForaCare Inc. is a provider of medical devices and telehealth software as a means to provide a disease management solution. Such existing telehealth systems can provide the building entry management system of the present invention data related to visitors wishing to enter the building and facilitate in granting or denying access to the building.

Another example of source of data can be https://pointclickcare.com/, which provides a cloud-based monitoring system for patient compliance and patient care. Such service can be coupled to the building entry management system of the present invention and provide information regarding visitors wishing to enter the building, and/or to stop all visitors from registering future visits.

According to the latter example the building entry management system can also provide visitors with information related to infected zones within the building. For example, when visitors arrive at a nursing home the computer kiosk at the entrance to the nursing home can alert the visitor based on census data drawn from pointclick care. This way, the visitor can be informed whether the area of the building he is visiting is an infected patient, allowing thereby the visitor to take necessary precautions.

According to an example, the method can further include providing each visitor with a contact tracing device, such as a wearable electronic device which tracks contacts of the visitor inside the building. The wearable electronic device can configured to detect and record physical presence in close proximity to another similar wearable electronic device. This, way, providing every person inside the building with the wearable electronic 20 device allow tracking any contacts of people inside the building. The wearable electronic device can be configured to allow determining the minimum distance between devices required for determining contact. For example, in case health officials determine that a disease can be transmitted between people disposed at a certain maximum distance from one another, the wearable electronic device can be configured to detect and record contacts at the predetermined distance or at shorter distances.

Each wearable electronic device is configured to record and store information regarding contacts with other similar devices. Each wearable electronic device is assigned an ID, such that when a contact is recorded, the ID of the device being contacted is recorded as well. This way, the wearable electronic device provides information regarding contacted devices and the people wearing these contacted devices can be identified.

The contacts of detected by the wearable electronic device can be stored in the visit log of each visitor, i.e., by reading the contact information accumulated inside the wearable electronic device. This way, in case a visitor was found to be infected, contacts of the visitor throughout the visit in the facility can be traced and people contacted the visitor can be provided with appropriate isolation and disease prevention instructions.

According to an example, the wearable electronic device can be further configured to measure or detect body vitals of the visitor during his/her visit, thereby alerting the visit of any change in his medical condition which may affect the access level permitted during the visit. Additionally, the wearable electronic device can be configured to facilitate social distancing between people within the facility, such as https://www.minew.com/product/B8-Plus/B8-Plus.html, which is configured to detect distances between corresponding wearable electronic devices. Furthermore, the facility can include a plurality of fixed presence sensors installed within the facility and being configured to detect the presence of wearable electronic devices. This way, the system can be configured to log specific areas of the facility visited by each of the visitors. This information can assist in contact tracing and facilitate mitigating future risks in case one of the visitors was found to be infected with a virus.

These sensors can also be utilized to guide visitors navigating in the facility. For example, the visitors can be provided with navigation information assisting them when finding their way within the facility. Such system is described in https://www.inpixon.com/, which is incorporated by reference herein, and can further be configured to integrate data related to infection risks. For example, in case a visitor is granted limited access due to a high infection risk, he/she may be provided with navigation information allowing them to reach the desired area in the facility while avoiding areas to which they are not granted access. The presence sensors can be configured to allow real-time navigation information, such as a dynamic map displayed on the visitor's handheld device, in accordance with the detected location of the visitor.

In addition, for visits which are restricted by time, the system can be configured to alert the visitor when the preset visit time is about to end. Such alert may be sent to the visitor's handheld device or the wearable electronic devices. The visitors can be required to check out of the facility, by scanning the access code, or by returning the wearable electronic devices when exiting the facility. This way, for visits which are restricted by time, the system can alert the facility manager or security system in case a visitor did not exit the facility after his visit time had expired.

Those skilled in the art to which the presently disclosed subject matter pertains will readily appreciate that numerous changes, variations, and modifications can be made without departing from the scope of the invention.

1. A method for managing building entry, the method comprising:
receiving from a visitor identification information;
determining geographical area of the building and obtaining local health information related to said geographical area;
obtaining medical data including at least one vital sign of said visitor;
calculating risk level of said visitor, based on said medical data and said local health information; and,
determining building access for the visitor based on said calculated risk level for said visitor.

2. The method in accordance with claim 1 wherein said at least one vital sign is selected from a group including body temperature, blood pressure, heart rate, respiratory rate, and oxygen level.

3. The method in accordance with claim 1 wherein said step of obtaining medical data further includes prompting said visitor to input data related to health condition.

4. The method in accordance with claim 1 further comprising obtaining locations data related to physical locations of said visitor during a predetermined period prior to said visit, and wherein said step of calculating risk level is carried out in accordance with said location data.

5. The method in accordance with claim 4 wherein obtaining said locations data includes receiving location information from a handheld device of said visitor.

6. The method in accordance with claim 1 wherein obtaining said medical data includes receiving an image of said visitor and extracting from said image said at least one vital sign.

7. The method in accordance with claim 1 further comprising determining visit characteristics of a visit by said visitor, said visit characteristics including a visit purpose.

8. The method in accordance with claim 7 wherein said visit characteristics includes data related to sections of the building to be visited during said visit.

9. The method in accordance with claim 1 wherein said step of providing said visitor with a building access information includes sending said access information to a handheld device of a user, wherein said access information is encoded.

10. The method in accordance with claim 1 wherein said access information includes access grant or denial and access restriction.

11. The method in accordance with claim 8 further comprising forming a visit log including said visitor identification information, visit characteristics, and access information.

12. The method in accordance with claim 11 wherein said step of calculating risk level includes obtaining information from visit logs of previous visits and calculating risk level of said visit in accordance with risk levels of said previous visits.

13. The method in accordance with claim 1 wherein said step of calculating risk level includes obtaining information from visit logs of previous visits and calculating risk level of said visit in accordance with risk levels of said previous visits.

14. The method in accordance with claim 1 further comprising tracking visiting locations of said visitor in the building during said visit and recording data of said visiting locations in said visit log.

15. A system for managing building entry, the system comprising:
a plurality of input devices configured for inputting identification information of a visitor and medical data related to at least one vital sign of said visitor;
an infection data module configured for receiving health information related to geographical area of a building to be visited; and
an access controller server configured to receive said identification information and said medical data, said access controller server is configured for calculating risk level of said visit in accordance with said medical data and said health information, said access controller server is further configured for providing said visitor with a building access information determined in accordance with said risk level.

16. The system of claim 15 further comprising a medical diagnostic tool for obtaining said at least one vital sign, and wherein said at least one vital sign is selected from a group including body temperature, blood pressure, heart rate, respiratory rate, and oxygen level.

17. The system of claim 15 further configured to determine visit characteristics of a visit by said visitor, said visit characteristics including a visit purpose, and wherein said calculating risk level is carried out in accordance with said characteristics.

18. The system of claim 15 wherein said plurality of input devices are configured for obtaining locations data related to physical locations of said visitor during a predetermined period prior to said visit, and wherein said access controller server is configured for calculating risk level in accordance with said locations data.

19. The system of claim 15 wherein said input devices are configured for obtaining an image of the visitor, and wherein the system further includes an image analyzer configured to extract from the image vitals of the visitor.

20. The system of claim 15 wherein said building access information includes encoded information related to access restriction.

21. (canceled)
